(19) United States
(12) Patent Application Publication
(10) Pub. No.: US 2011/0287440 A1
(43) Pub. Date: Nov. 24, 2011

(54) Composition and method for reconstituting IkB kinase in yeast and method of using same

(75) Inventors: Ebrahim Zandi, Duarte, CA (US); Beth Schomer Miller, Los Angeles, CA (US)

(73) Assignee: University of Southern California

(21) Appl. No.: 13/136,467

(22) Filed: Aug. 1, 2011

Related U.S. Application Data
(63) Continuation of application No. 10/079,949, filed on Feb. 19, 2002, now abandoned.
(60) Provisional application No. 60/269,499, filed on Feb. 16, 2001.

(51) Int. Cl. C12Q 1/68; C12N 9/96; C12Q 1/48; G01N 33/573; C12N 9/12; C12N 1/19

(52) U.S. Cl. 435/6.18; 435/194; 435/254.2; 435/15; 435/7.4; 435/188

(57) ABSTRACT

The invention provides a means for reconstituting IκB kinase in yeast in order to study the structure and regulation of IKK and to produce pharmacological therapies to block IKK. This invention can be used to express an IKK complex that is biochemically identical to IKK isolated from native cells and in coupled in vitro kinase assays to screen for its upstream regulators. The IKK expressed by reconstituting the yeast can be used to screen for unknown substrates and for pharmacological therapies that block its activity. The invention could also be used to screen for compounds that enhance its activity. The IKK may also be used as a source of material for crystallization and X-ray diffraction analysis.

### CROSS-REFERENCE TO RELATED APPLICATIONS

This application claims the benefit of U.S. Provisional Application No. 60/269,499, filed Feb. 16, 2001, the entire disclosure of which is hereby incorporated by reference in its entirety for all purposes.

### FIELD OF THE INVENTION

The present invention relates to molecular biology and biochemistry and, in particular, to reconstituting IκB kinase (“IKK”) in yeast and using IKK expressed in yeast.

### BACKGROUND OF THE INVENTION

### A. Cellular Function of IKK Kinase

Upon cellular exposure to stimuli, such as an infection or stress, nuclear factor κB (“NF-κB”) transcription factor triggers gene expression. For example, when a cell is subjected to an infection, within minutes NF-κB triggers vasodilation and infiltration of macrophages. Under normal circumstances, the NF-κB transcription factor is tightly regulated to allow an appropriate and rapid response to infection or stress while preventing an inappropriate inflammation from a false trigger. Misregulation of NF-κB, however, can cause uncontrolled expression of inflammation-causing genes and contributes to the pathogenesis of a number of diseases including rheumatoid arthritis, bronchial asthma, inflammatory bowel disease, septic shock, adult respiratory distress syndrome, and transplant rejection. It also plays a role in autoimmune diseases including diabetes. In rheumatoid arthritis, for example, activation of NF-κB causes release of inflammatory mediators including prostaglandins, thromboxanes, and leukotrienes (26), leads to release of adhesion molecules which may allow the leukocytes to interact with synoviocytes, and stimulates production of IL-6, IL-8 and GM-CSF (29). Finally, NF-κB induces further production of TNF-α and IL-1 leading to a feedback loop amplification of the inflammation response.

Activation of NF-κB is also associated with cancer. Virally encoded gene products, protein X from hepatitis B and tax from human T-cell leukemia virus activate NF-κB and other transcription factors and cause improper cell proliferation (10, 21). In addition, TNF and NF-κB contribute to skeletal muscle decay known as cachexia (11) which accounts for one-third of cancer mortalities with inflammatory origin.

There is also a rare X-linked disorder known as incontinentia pigmenti which is caused by deficient IKKγ expression (18, 28). This condition is usually prenatally lethal in males, but in females, defective NF-κB activation causes abnormalities in skin, hair, nails, teeth, eyes, and the central nervous system.

NF-κB also stimulates production of proteins that prevent apoptosis and regulate the cell cycle. The anti-apoptotic function of NF-κB is clearly evidenced by knock-out experiments where the absence of multiple forms of NF-κB (5) or its upstream regulators (16, 28) results in embryonic lethality due to massive liver apoptosis. Rel/NF-κB activates the expression of anti-apoptotic genes including Bcl-X and Bf1/A1 (13) and inhibitors of apoptosis (IAPs) (33, 40). This promotes survival from apoptosis normally triggered by TNF or chemotherapeutic agents (4, 5, 17, 34-37). In some cells, NF-κB also stimulates apoptosis (9).

In resting cells, NF-κB is found predominantly in the cytoplasm in a complex with IKK, an inhibitory subunit, which sequesters NF-κB and prevents its migration to the nucleus (2). Diverse stimuli lead to phosphorylation of two serine residues, S32 and S36 on IκBα, which targets IκBα for polyubiquitination and proteolytic degradation. This frees NF-κB to move to the nucleus where it binds with high affinity to KB elements in the promoter region of target genes (32).

NF-κB comprises a family of dimeric transcription factors that regulate the expression of over 150 genes—most of which are necessary to respond to infection, injury, and physical stresses (24). Genes induced by NF-κB include cytokines, immunoreceptors, immunoglobulins, leukocyte adhesion proteins, nitric oxide synthase, and COX2. NF-κB is activated by a variety of factors, including mitogens such as phorbol ester, viral and bacterial products (such as lipopolysaccharide) (31), cytokines such as tumor necrosis factor-α and interleukin-1 (23) and stresses such as hydrogen peroxide (30) and ionizing radiation (6).

A diverse array of signals traverse multiple signaling pathways to stimulate NF-κB. All the signaling pathways, with the exception of ultraviolet radiation (15), converge at a specific critical regulatory point: the phosphorylation of amino terminal serines on IκB. This phosphorylation is catalyzed by a large kinase complex, IκB kinase (“IKK”) (8, 20, 44). Because phosphorylation of IκBα by IKK is the key step in activation of NF-κB, understanding the structure and regulation of the complex is critical and, as discussed below, could be used to develop therapies to treat a variety of inflammatory and autoimmune diseases.

### B. Research on the Structure, Function, and Regulation of IKK

Some aspects of the structure of IKK are known in the art (43). In 1997, a research group, including one of the inventors of the present invention, isolated and sequenced IKK. (See U.S. Pat. No. 6,242,253). IKK is composed of two homologous kinase subunits IKKα and IKKβ (85 kD and 87 kD respectively) and a 52 kD regulatory subunit IKKγ (8, 42, 44). The α and β subunits are associated with each other via their leucine zippers (42). It is believed that an α-helical regions towards the N-terminus of IKKγ interacts with six amino acids at the C-terminus of IKKα and IKKβ (19). IKKγ is required for activation of IKK in response to TNF and other stimuli (27). Experiments show that interrupting this interaction leads to a higher basal IKK activity but prevents stimulation of IKK by TNFα (19). Recombinant IKKγ forms dimers and trimers (27), and it is possible that IKKγ mediates formation of the large IKK complex.

A number of molecules in the TNFα signaling pathway have been shown to be involved in activation of IKK by TNF-α and by IL-1. The binding of a ligand to a receptor induces receptor trimerization and subsequent recruitment of signaling proteins including TRADD, RIP, and TRAFs (which also trimerize). Forced oligomerization of these signaling proteins stimulates the downstream effects (3), suggesting that this is a key event in IKK activation. Experiments have indicated that the IKK complex (through IKKγ) interacts with RIP and the TNF receptor after stimulation (45).

NF-κB can be inhibited by pharmacological agents that block its activation at various steps. Glucocorticoid therapy is used to treat rheumatoid arthritis, asthma, and inflammatory bowel disease and to prevent transplant rejection (14). Glucocorticoids inhibit NF-κB by enhancing the production of inhibitors of NF-κB (1) and by blocking the transactivation of NF-κB in the nucleus (25). Glucocorticoids are somewhat effective therapies, but their side-effects necessitate development of more specific drugs. Non-steroidal anti-inflammatory drugs are also widely prescribed for treating inflammatory diseases. Sulfasalazine (38) and aspirin and salicylate (39) exert their anti-inflammatory effects in part by directly inhibiting IKK activity; all of these agents compete with ATP for binding IKK. However, the efficiency of these agents in blocking NF-κB is weak and non-specific, and all of them have other pharmacological effects producing deleterious side effects which limit dosages sufficient to block NF-κB (14).

There is a great need for a better understanding of the structure and regulation of IKK in order to produce pharmacological therapies to block IKK and develop treatments for inflammatory and autoimmune diseases as well as cancer.

### C. Methods of Producing and Isolating IKK

In order to effectively study the structure, function and regulation of IKK, efficient means for producing and isolating IKK must be employed. Bacteria, Sf9 cells, and mammalian cell culture have been described for the production and isolation of IKK, but each has substantial drawbacks. For example, IKK expressed in bacteria forms large aggregates which are not native or functional.

IKKα and IKKβ have been expressed alone and in combination in Sf9 cells (12, 42). However, in the baculovirus system, co-expression of two or all three subunits of IKK is technically difficult. In addition, insect cells contain IKK-related proteins and signaling pathways that can activate IKK (42).

Sf9 and mammalian systems also have the disadvantages of endogenous IKK and redundant factors that are not found with yeast. Studying signal transduction directly in mammalian or other higher eukaryotic cells is difficult because many signaling pathways have similar and redundant factors, and many of the signal transduction pathways intersect and act upon each other. Because IKK is a large complex composed of three different subunits, there may be multiple complexes of IKK which exist to respond to different signals. Furthermore, IKK responds to over 150 signals, studying IKK in mammalian cells is particularly difficult.

A popular method in signal transduction research is to use mammalian cell culture, such as HeLa cells or mouse embryonic fibroblasts, to overexpress a wild type or dominant negative protein in and to test for any effect on IKK activity. Cell culture can be used to show if a protein at least has the potential to be involved in a given network, but it has inherent problems. First, it is difficult to determine if the overexpressed protein acts directly or indirectly. Second, when an enzyme or regulatory protein is expressed at higher than normal levels, it may associate with proteins and networks where it may not normally localize. As a result, the protein may act non-specifically and yield misleading results.

Mechanistic analysis is also complicated in both Sf9 and mammalian cells due to endogenous proteins. For example, there may be multiple existing IKK complexes in a given cell. Expressed mutated forms of IKK are directed into heterocomplexes containing endogenous proteins, and the effect of the mutation may be lost in a background of these endogenous proteins (44). In some situations, one might try to overcome this by expressing the mutated form at very high levels, but this often results in obscured results due to non-specificity. For example, proteins expressed in cells can interact with non-physiological partners due to the weak similarities in the interaction domains that exist among many proteins of similar functions and results in non-specific activating or inhibiting effects.

As shown in U.S. Pat. No. 6,312,923, transducing yeast cells to express functional recombinant proteins is known in the art, the present invention was the first to succeed in reconstituting IKK Complex in yeast. Because it was believed that yeast expressed a protein system analogous to mammalian IKK, those skilled in the art believed it would not be possible to reconstitute IKK in yeast. However, because the present inventors found that yeast does not express these proteins, they were able to develop disclosed methods and compositions.

Transforming yeast cells to express IKK overcomes these difficulties inherent in bacteria, Sf9, and mammalian cell culture. Yet there are very few endogenous kinases that have been identified in yeast resulting in a very low background of endogenous activity. Because yeast are eukaryotic, they can produce native and functional enzyme. One can also transform a given yeast cell with multiple plasmids, each plasmid with a different selection marker and the gene for a different subunit of IKK. With yeast, it is also possible to isolate strains that consistently co-express multiple subunits of IKK.

Furthermore, a biochemical approach to learning about IKK allows the a single variable at a time to be addressed. The inventors have discovered that yeast cells, S. cerevisiae in particular, lack TNF-α and NF-κB signaling pathways. Therefore, exogenously proteins expressed by the yeast cell do not affect the IKK signaling pathways. Because the only IKK expressed by the transformed yeast cell is from the plasmid insert, it is simple to test if a single molecule or subcellular fraction mutation affects the activity of the enzyme. IKK expressed in yeast can be used for clean mechanistic analysis. It can also be used to study the composition of the enzyme complex and how it is regulated.

The heterologous expression of IKK allows the examination of the composition of the enzyme complex and how it is regulated. It allows for very clean mechanistic analysis that facilitates, for example, designing experiments on the function of specific residues or domains. In addition, by co-expressing putative upstream regulators, partial or entire signal transduction cascades can be reconstituted in a heterologous, in vivo, system without interference from endogenous molecules.

### SUMMARY OF THE INVENTION

The present invention provides a method for reconstituting IKK in yeast and the resulting composition of reconstituted IKK complex. Accordingly, the present invention provides a means to study the structure and regulation of IKK and to produce pharmacological therapies to block IKK.

In a first aspect of the invention, a novel method for reconstituting IKK in yeast is provided, said method comprising the steps of (1) subcloning the genes for the three subunits of IKK into separate vectors, each with a different selection marker, tags, and an inducible promoter, and transforming the vectors alone or in combination into yeast; (2) growing the yeast in selective liquid media; (3) controllably inducing the proteins by means of inducible promoters; (4) lysing the yeast, (5) extracting the IKK; and (6) purifying the IKK.

It is another object of the present invention to provide a composition of IKK reconstituted in yeast. In a related aspect, the invention is an IKK complex that is biochemically identical to IKK isolated from wild type cells.

In yet another aspect of the invention, a mechanism for the regulation of the IKK complex is disclosed wherein IKKγ regulates the autophosphorylation of the T loop residues in the kinases domain of IKKβ. When the T loop residues are phosphorylated, the kinase is active. This phosphorylation is required for activation of the IKK complex. In the inactive state, the T loop residues are not phosphorylated while the γBD serine(s) are phosphorylated, and the phosphorylation of the serine(s) prevents IKKγ from facilitating self-activation. The activation of the complex requires dephosphorylation of these γBD serines, which then allows IKKγ to facilitate autophosphorylation of IKKβ in the T loop.

In another aspect, the present invention pertains to using the IKK expressed in yeast to develop drug and diagnostic therapies. The present invention can be used in coupled in vitro kinase assays to screen for its upstream regulators and can also be used in assays to screen for unknown substrates. It can be used to screen for pharmacological therapies to block its activity; likewise it can be used to screen for compounds that enhance its activity. IKK expressed in yeast may also be a good source of material for crystallization and X-ray diffraction analysis.

The details of the preferred embodiments of the present invention are set forth in the accompanying drawings and the description below. Once the details of the invention are known, additional innovations and changes will become obvious to one skilled in the art.

### BRIEF DESCRIPTION OF THE DRAWINGS

FIG. 1. Western blot analysis of human IKK expressed in yeast

The expression of the reconstituted IKK proteins was assessed by 3 parallel Western blots. The top blot was probed with a monoclonal antibody against IKKβ. The middle blot was probed with monoclonal antibodies directed against IKKα and IKKγ. The bottom blot was probed with monoclonal antibodies directed against HA.

The yeast contained vectors for α only, β only, β and γ, α and γ, α, β, and γ, and α, β (dominant negative) and γ, as indicated. The far right lane “YPD” denotes the parental strain into which no plasmids were transformed.

FIG. 2. Human IKK expressed in yeast forms a large complex similar to IKK from HeLa cells

HeLa cells were stimulated with 10 ng/ml TNF for 10 min and lysed, and the 65,000 g supernatant was separated by Q column fractionation. The peak activity was applied to a superose 6 gel filtration column and chromatographed. The IKK activity in each fraction is shown; the peak activity elutes at ˜900 kD.

Yeast expressing human IKKα, IKKβ, and IKKγ were lysed, and the 65,000 g supernatant was applied to the superose 6 gel filtration column and chromatographed. IKKαβγ activity eluted as a large complex (similar to IKK from HeLa cells). IKK activity from αβγ-expressing yeast was also seen in smaller complexes; most likely these complexes contain the catalytic subunits as dimers without IKKγ. The activity from yeast expressing IKKβ only elutes as a small 158-230 kD complex.

FIG. 3. Activity of reconstituted IKK in yeast

IKK from HeLa cells was partially purified by Q fractionation followed by Superose 6 gel filtration.

As previously shown, stimulation with TNF caused an increase in IKK activity in the HeLa cells: non-stimulated (lanes 7-9) or stimulated with 10 ng/ml TNF for 10 min (lanes 10-12).

Yeast were transformed and IKK was partially purified by Superose 6 gel filtration. Lane 1 shows the activity of yhIKKβ. Lanes 2-4 show the activity for increasing amounts of yhIKKβγ, and lanes 5-6 show the activity for increasing amounts of yhIKKαβγ. YhIKKβγ and yhIKKαβγ had higher activity per amount of IKKβ than TNF-stimulated HeLa IKK. The activity of yhIKKβ was similar to that of TNF-stimulated HeLa cells.

For kinase assays, the reaction contained 20 mM Tris-HCl (pH 7.6), 20 mM MgCl₂, 20 μM cold ATP, 2 mM DTT, 33 μg/ml GST-IκBα₁₋₅₄, 167 μCi/ml γ³²P ATP, and varying amounts of extract as shown. Following PAGE and transfer, the membrane was exposed to phosphoimager (top panel) before Western blot analysis was done to compare levels of IKKβ (bottom panel).

FIG. 4. The activity of IKK is negatively regulated by phosphorylation of its gamma binding domain.

Regulatory serines (177 and 181) in the kinase domain (T loop) and putative regulatory serines (740 and 750) in the gamma binding domain (γBD) of IKKβ were mutated to alanines in order to prevent any phosphorylation and/or to glutamic acids in order to mimic the charge in the phosphorylated state. These mutated forms of IKKβ were expressed in yeast with and without IKKγ, and the complexes were partially purified from the lysates by superose 6 gel filtration. The kinase activity of the IKK complexes towards GST-IκBα was assessed as in FIG. 3.

Lane 1 shows the activity of IKKβ alone. Lane 2 shows that IKKβ reconstituted with IKKγ had higher activity than IKKβ alone, indicating that IKKγ facilitates self-activation of IKK. In lane 3, the T loop regulatory serines is mutated to glutamic acids to mimic phosphorylated state results in high activity which is not further activated by co-expression with IKKγ (lane 4) indicating that the regulatory role of IKKγ is upstream to the phosphorylation of the T loop serines. In lane 5, the T loop serines are mutated to alanines and completely inactived the kinases. As seen in lane 6, this mutated IKKβ T loop AA is not activated at all by the presence of IKKγ. When the serine residues in the gamma binding domain are mutated to glutamic acid, IKKβ still has a low level of kinase activity (see lane 7), similar to wild-type IKKβ (see lane 1).

However, as shown in lane 8, reconstitution with IKKγ does not allow IKKβγBDEE to become activated. This result indicates that the analog of phosphorylated serines in the gamma binding domain prevents IKKγ from facilitating self-activation of IKK and suggests that phosphorylation of these amino acids is a mechanism to maintain IKK in an inactive state. In lane 9, IKKβ with the 6 amino acids in the γBD domain at the C terminus (LDWSWL) were deleted and subsequently had a low level of activity, similar to wild-type IKKβ. But, as seen in lane 10, IKKγ could not allow the complex to self-activate. This result indicates that the γBD is required for self-activation of the complex.

Lane 11 shows the activity of IKKβAA in which 3 serines at the γBD (733, 740, 250) were mutated to alanine; this mutant had a similar level of IKK activity to wild-type, and, as seen in lane 12, IKKγ facilitates its self-activation similarly to wild-type. This result indicates that IKKγ is able to facilitate self-activation of wild-type or mutated IKKβAA, but when these key residues are mimicking phosphorylation as with IKKβEE, the self-activation is prevented.

Finally, when both the T loop serines and γBD serines are mutated to glutamic acids to mimic phosphorylation, the kinase has a high activity (see lane 13), and its activity is not further enhanced by the presence of IKKγ (see lane 14). This result indicates that once the T loop residues are phosphorylated, the kinase activity will be high, regardless of the status of phosphorylation of the γBD or the presence of the IKKγ in the complex.

FIG. 5. Detection of phosphorylated IκBα in yeast by phosphoantibodies.

Ten micro gram proteins in yeast extracts expressing IIKKβ:IKKγ along with either IKKβAA (AA lane), IκBα (wt lane), or vector (NO) were separated on duplicates 13% SDS page and transferred to PVDF membrane. Blots were probed either with anti-phospho-IκBα (left), or with anti-IκBα (right).

### DETAILED DESCRIPTION OF THE INVENTION

All scientific terms are to be given their ordinary meanings as understood by those of skill in the art, unless an alternate meaning is set forth below. In case of conflict, the definitions set forth in this specification shall control.

As used herein, the term “isolated,” when used in reference to an IKB kinase complex or to an IKK subunit of the invention, means that the protein complex or subunit is relatively free from contaminating lipids, proteins, nucleic acids or other cellular material normally associated with an IKK in a cell.

The proteins of the invention include heterologously expressed and functional that are substantially identical to naturally occurring forms as well as mutations thereof. These variants will be substantially homologous and functionally equivalent to the native protein. A variant of a native protein is “substantially homologous” to the native protein when at least about 80%, more preferably at least about 90%, and most preferably at least about 95% of its amino acid sequence is identical to the amino acid sequence of the native protein. A variant may differ by as few as 1, 2, 3, or 4 amino acids. By “functionally equivalent” is intended that the sequence of the variant defines a chain that produces a protein having substantially the same biological activity as the native protein of interest.

The IKK subunits have binding sites with high affinity for specific signaling molecules. By measuring the biological activity of the reconstituted kinase, the functional equivalence can be determined. Biological activity is measured using assays specifically designed for measuring the activity of the native IKK, including the assays described in the present invention. In addition, antibodies raised against the biologically active native IKK protein can be tested for their ability to bind to the functionally equivalent variant. Binding indicates that the protein has a conformation similar to the native IKK.

The disclosed method for reconstituting IKK kinase involves the coexpression in yeast cells of genes encoding all three subunits of the IKK kinase complex, IKKα, IKKβ and IKKγ. The subunits can be introduced in the yeast strain on a stable plasmid (e.g. pESC), or it can be integrated into the yeast chromosome using standard techniques (46).

The choice of expression vectors for use in connection with the IKK subunits is not limited to a particular vector. Any expression vector suitable for use in yeast cells, including small yeast chromosomes (“YACs”) and cosmids, is appropriate. The discussion related to experiments in the Examples section below describes particular vector, promoter and tag combinations that yielded meaningful results. However, many options are available for genetic markers, promoters and ancillary expression sequences. As discussed in greater detail below, the use of an inducible promoter to drive expression of the cDNA library is a preferred feature which provides a convenient means for demonstrating that observed changes in IKK activity are, in fact, dependent on the cDNA library derived from the IKK complex subunits. Likewise, the inclusion of a tag, or a small polypeptide, facilitates determining the ratio of alpha to beta to gamma subunits were expressed by the transposed yeast cells.

Likewise, the choice of yeast cells is not limited to Saccharomyces cerevisiae. Other species in the Pichia genus as well as other genera of yeast cells may also be appropriate host cells.

In the present invention, the cDNA library is prepared by isolating mRNA of IKKα, IKKβ, and IKKγ. An RNA-directed DNA polymerase is employed for first strand synthesis using the mRNA as a template. Second strand synthesis is carried out using a DNA-directed DNA polymerase which results in the cDNA product. Following conventional processing to facilitate cloning of the cDNA, the cDNA is inserted into an expression vector suitable for use in yeast cells. Preferably the promoter which drives expression from the cDNA expression construct is an inducible promoter. The cDNA expression library is then used to transform the yeast strain which constitutively expresses the IKKα, IKKβ, or IKKγ subunits.

The preferred method for determining the level of intracellular IKK phosphorylation is a colony Western blot using replica plates. It will be recognized that, although particularly convenient, the colony Western blot is but one example of many conventional assays which could be employed to determine levels of intracellular IKK kinase activity. In the colony Western blot procedure, cDNA library transformants are initially plated on media which do not contain an inducer of the promoter which drives expression of the cDNA insert. For example, if the GAL1 promoter is used to drive expression of the cDNA insert, the cDNA library transformants are initially plated on a medium containing 2% glucose. On this growth medium, cells containing the cDNA expression will grow, but the encoded cDNA product is not expressed. In order to express the encoded cDNA, the cDNA library transformants must be plated on a medium containing galactose. As another example, in the present invention, IKKα and IKKβ were subcloned into pESC ura and pESC trp vectors in which the galactose promoter region was replaced with the met promoter from the leu(met) vector. In these plasmids, the presence of methionine represses expression of IKK, but expression is induced by removal of the methionine.

A set of replica filters is produced from the initial transformation plate by sequentially placing a set of directionally oriented membranes (e.g. nitrocellulose filter membranes) over the transformation plate such that the membrane contacts existing transformant colonies. Cells from transformation colonies adhere to the membranes to form a pattern which represents the pattern of colonies on the transformation plate. Each of the replica filters is then placed on a separate plate, one of which contains a compound which will induce the inducible promoter (e.g. 2% galactose to induce the GAL1 promoter) and one which will not induce the inducible promoter (e.g., 2% glucose for the GAL1 promoter). Both plates are incubated overnight to promote regrowth of the original cDNA library transformants.

Following overnight incubation, the replica filters are removed from the growth medium plates, and the colonies are lysed in situ by soaking the replica filters in a lysis solution for a period of time sufficient to lyse cellular membranes. A number of different solutions are used for lysing cellular membranes, the formula described in the examples below are just one of many that could be used. The replica filters are then probed with monoclonal antibodies directed against the IKKα, IKKβ, and IKKγ subunits as well as the tag HA. Colonies which exhibit elevated IKK kinase activity on the replica filter which had been incubated overnight on a growth medium containing a compound which induces expression of the cDNA insert linked to the inducible promoter.

The method of the present invention is not limited to the reconstitution of IKK complex in yeast. Rather, the method can be modified for use reconstituting a number of different kinases and proteins—especially those composed of multiple subunits.

The following examples are intended to illustrate but not limit the present invention. The methods of the present invention can be further modified for uses such as the identification of drug and diagnostic therapies.

### Example I

This example provides a method for reconstituting and isolating IKK in yeast cells.

cDNA library sequences of the three subunits of IKK, γ, α, and β, were subcloned into Strategene™ pESC expression vectors with different promoters and selection markers. Each subunit has a promoter (e.g. galactose or alcohol dehydrogenase), a different selection marker (e.g. leucine, histidine, or tryptophan), and a tag (e.g. myc, HA, or FLAG). IKKα and IKKβ were subcloned into pESC ura and pESC trp vectors in which the galactose promoter region was replaced with the met promoter from the leu(met) vector. In these plasmids, the galactose (gal) promoter regulates the gene so the protein is only expressed when the yeast are induced with galactose. Likewise, with the methionine promoter, the presence of methionine represses expression of IKK, but expression is induced by removal of the methionine. Yeast were also transformed with plasmids in which the methionine (met) promoter regulates expression of IKK (7, 22). For IKKγ, the cDNA library was subcloned into a pESC 86(+) expression vector which induces constitutive expression under the alcohol dehydrogenase (ADH) promoter or was directly cloned into the leu(met) vector. Examples of plasmids and yeast strains used in the present invention are shown in Tables 1 and 2 respectively.

The plasmids containing the different subunits of IKK are transformed into a yeast cell using lithium acetate as described in the Stratagene pESC Yeast Epitope Tagging Vectors Instruction Manual. Each yeast cell can be transformed with multiple copies of plasmids containing multiple copies of plasmids containing IKKα, IKKβ, or IKKγ sequences.

A 2 mL overnight culture of yeast, grown in non-inducing drop out medium, is added to 400 mL of appropriate non-inducing drop out medium. The yeast are grown at 30° C. with shaking at 300 rpm for approximately 30 hours before being transferred to the inducing medium for 10-12 hours at 30° C. with shaking.

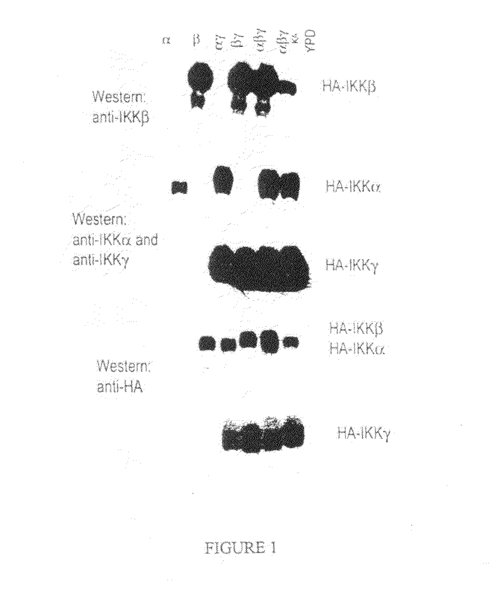

For harvesting and lysing the yeast, all steps are performed at 4° C. unless otherwise indicated. The yeast are first washed in 400 mM (NH₄)₂SO₄, 200 mM Tris-HCl, (pH 8.0), 10 mM MgCl₂, 10% glycerol containing yeast protease inhibitors (2.5 μg/ml leupeptin, 20 μg/ml aprotinin, 2.5 μg/ml antipain, 2 μg/ml pepstatin, 1 mM PMSF, 0.1 μg/ml chymostatin, and 1.1 μg/ml phosphoramidon). 1-2 g of yeast pellet are resuspended in 2 mL lysis buffer (20 mM Tris (pH 7.6), 20 mM NaF, 20 mM 13-glycerophosphate, 0.5 mM Na₃VO₄, 2.5 mM sodium metabisuphite, 5 mM benzamidine, 1 mM EDTA, 0.5 mM EGTA, 10% glycerol, 300 mM NaCl, 1% triton X-100 with yeast protease inhibitors) in a capped 15 mL conical tube, frozen at −80° C., and thawed on ice. Acid-washed 425-600μ glass beads (equal in volume to the yeast pellet) are added to the yeast, and the suspension is vortexed for 3×1 min (with 1 min incubation on ice between mixings). The suspension is then centrifuged at 3000 g for 3 min, and the supernatant is collected. To extract more protein, 1 ml additional lysis buffer is added to the yeast, and the vortexing and centrifugation and resuspension procedure is repeated an additional 8 times. To remove particulate material, the crude supernatant is centrifuged at 65,000 g for 1.5 hr, and the supernatant is collected and stored at −80° C. For some experiments, IKK is partially purified using superose 6 gel filtration chromatography as described (44). The disclosed invention expresses an IKK complex that appears biochemically identical to IKK isolated from wild type cells. FIG. 1 shows a Western blot of yeast strains expressing HA-IKKα, HA-IKKβ, HA-IKKβ_{KA} (dominant negative) and HA-IKKγ alone and in combination. The subunits for IKK were seen only in strains transformed with these genes.

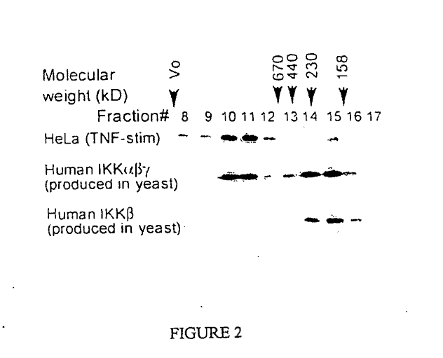

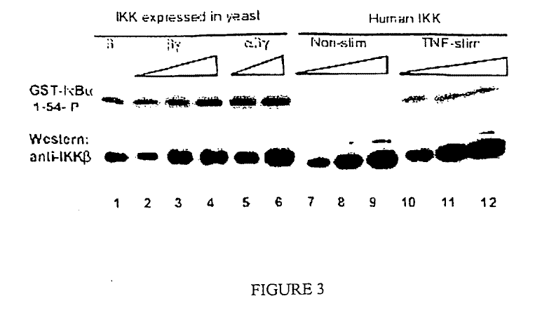

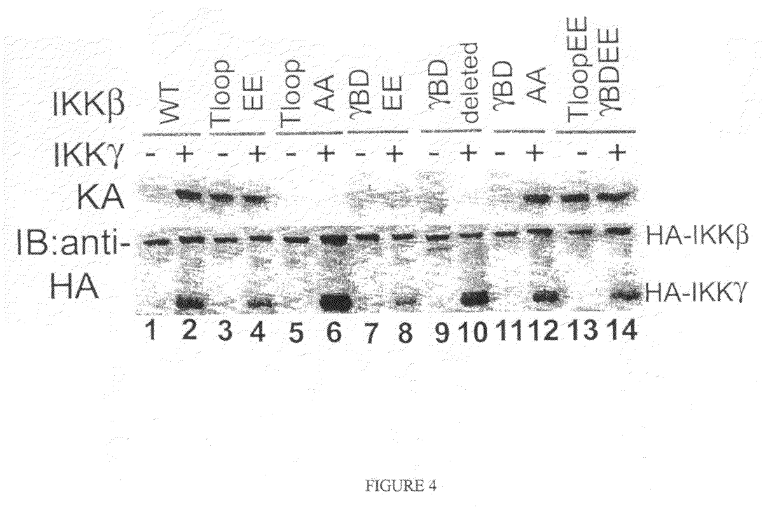

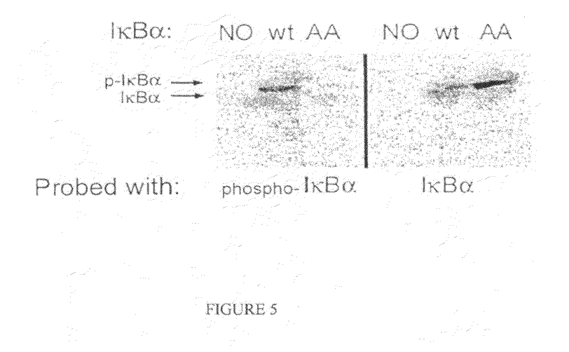

IKK produced in yeast (yhIKK) forms a large complex like IKK from HeLa cell lysates as evidenced by gel filtration chromatography. As shown in FIG. 2, both IKK activity from HeLa cells and IKK activity from yeast expressing α, β, and γ elute from a superose 6 gel filtration column as a large ˜900 kD complex. This result indicates that the yhIKKαβγ we have expressed is native and that, most likely, the 900 kD complex contains no additional proteins. When only the catalytic subunit is overexpressed in mammalian cells, IKKγ is limiting and the catalytic subunit (without the IKKγ subunit) elutes as an apparent dimer at 158-230 kD. A 158-230 kD complex is also seen in the yeast expressing α, β, and γ, and this is probably due to an excess of catalytic subunits over IKKγ; IKK activity from yeast expressing IKKβ only also elutes at 158-230 kD.

Human IKK activity from non-stimulated or TNF-stimulated HeLa cells was compared to yhIKK (β, γγ, and αβγ). yhIKK (αβγ or βγ) activity was found to be slightly higher than the activity from TNF-stimulated HeLa cells. Human IKKβ expressed in yeast had a level of activity similar to TNF-stimulated HeLa cells. In these experiments IKK levels were similar in the yeast and HeLa extracts as assessed by Western blot as shown in FIG. 3. The heterologously expressed IKK comples is substantially homologous to IKK isolated from mammalian cells.

### Example II

This example provides a method for identifying mechanisms for the regulation of the IKK complex. For example, by mutating to alanines (to prevent any phosphorylation) and/or to glutamic acids (to mimic the charge in the phosphorylated state) regulatory serines (177 and 181) in the kinase domain (T loop) and putative regulatory serines (740 and 750) in the gamma binding domain (γBD) of IKKβ and then expressing and partially purifying these mutated forms of IKKβ in yeast with and without IKKγ, the following mechanism for the regulation of the IKK complex is suggested: IKKγ regulates the autophosphorylation of the T loop residues in the kinases domain of IKKβ. This phosphorylation is required for activation of the IKK complex. In the inactive state, the T loop residues are not phosphorylated while the γBD serine(s) are phosphorylated, and the phosphorylation of the serine(s) prevents IKKγ from facilitating self-activation. The activation of the complex requires dephosphorylation of these γBD serines, which then allows IKKγ to facilitate autophosphorylation of IKK in the T loop. When the T loop residues are phosphorylated, the kinase is active.

In this experiment, the regulatory, serines (177 and 181) in the T loop and putative regulatory serines (740 and 750) in the gamma binding domain γBD of IKKβ were mutated to alanines in order to prevent phosphorylation and/or to glutamic acids in order to mimic the charge in the phosphorylated state. These mutated forms of IKKβ were expressed in yeast with and without IKKγ. The complexes were partially purified from the lysates by superose 6 gel filtration. The kinase activity of the IKK complexes towards GST-IκBα was assessed as in FIG. 3.

As illustrated in FIG. 4, IKKβ reconstituted with IKKγ (lane 2) had higher activity than IKKβ alone (lane 1), indicating that IKKγ facilitates self-activation of IKK. Mutating the T loop regulatory serines to glutamic acids to mimic phosphorylated state results in high activity (lane 3) which is not further activated by co-expression with IKKγ (lane 4). This result indicates that the regulatory role of IKKγ is upstream to the phosphorylation of the T loop serines.

Mutation of the T loop serines to alanines makes the kinase completely inactive (lane 5), and this mutated IKKβ T loop AA cannot be activated at all by the presence of IKKγ (lane 6). When the serine residues in the gamma binding domain are mutated to glutamic acid, IKKβ still has a low level of kinase activity (lane 7), similar to wild-type IKKβ (lane 1). However, reconstitution with IKKγ does not allow IKKβγBDEE to become activated (lane 8). This result indicates that the analog of phosphorylated serines in the gamma binding domain prevents IKKγ from facilitating self-activation of IKK and suggests that phosphorylation of these amino acids is a mechanism to maintain IKK in an inactive state.

IKK with the 6 amino acids in the γBD domain at the C terminus (LDWSWL) deleted had a low level of activity (lane 9), similar to wild-type IKKβ, but IKKγ could not allow the complex to self-activate (lane 10). This result indicates that the γBD is required for self-activation of the complex.

Lane 11 shows the activity of IKKβAA in which 3 serines at the γBD (733, 740, 250) were mutated to alanine; this mutant had a similar level of IKK activity to wild-type, and IKKγ facilitates its self-activation similarly to wild-type (lane 12). This result indicates that IKKγ is able to facilitate self-activation of wild-type or mutated IKKβAA, but when these key residues are mimicking phosphorylation as with IKKβEE, the self-activation is prevented.

Finally, when both the T loop serines and γBD serines are mutated to glutamic acids to mimic phosphorylation, the kinase has a high activity (lane 13), and its activity is not further enhanced by the presence of IKKγ (lane 14). This result indicates that when the T loop residues are phosphorylated, the kinase activity will be high, regardless of the status of phosphorylation of the γBD or the presence of the IKKγ in the complex.

### Example III

In another embodiment, the present invention can be used to develop a method for assaying IKK activity in situ in yeast. The present invention provides two methods for using the present invention to screen for upstream regulators of IKK. First, because the IKK complex reconstituted in yeast is only partially active, the protein extracts from cytokine stimulated mammalian cells can activate the IKK complex in coupled in vitro kinase assays. Such activity can be then purified biochemically to identify the protein components of it. Second, as described below in EXAMPLE IV, a system can be established to isolate potential negative regulators in situ in yeast. The in situ system is based on determining the activity of IKK in yeast by assessing an antibody that recognizes only the phosphorylated form of IκBαser 32.

To test the principle of the detection in the system, which is based on in situ identification of phosphorylated IκBα, full length HA-IκBα wild type or HA-IκBαAA, in which the two regulatory serines, 32 and 36, at the amino terminus are mutated to alanines [39], was transformed into S. cerevisiae that express IKKβ:IKKγ. Clones expressing these proteins were selected and examined by Western blot for the expression of proteins and phosphorylation of IκBα using phosphoantibodies (Cell Signaling 5A5 monoclonal antibody. As indicated by the data shown in FIG. 5, IκBα wild type, but not IκBαAA, is phosphorylated in cells expressing IKKβ:IKKγ.

As indicated by these assays, IκBα is phosphorylated in yeast by IKKβ:IKKγ complex and the phosphorylation can be detected by phosphoantibodies to IκBα. IκBα was not phosphorylated in yeast that does not express IKK complex or expresses kinase defective IKKβ:IKKγ.

The next step is to use the yeast strains shown in Table 2 and a control yeast for in situ detection of the phosphorylated IκBα. The yeast are plated and grown at 30° C. on agar plates containing an appropriate selective non-inducing medium and the replica are plated. The yeast are then grown to a sufficient density on membranes in plates over non-inducing media. Next, the IKK is induced by switching the membrane to a dish containing selective media without met and incubating for four hours. The yeast is fixed on the membrane with 4% paraformaldehyde for 90 min at room temperature. The membrane is washed 3 times with 0.1 M potassium phosphate (pH 6.5) and then the cell wall is dissolved with β-glucuronidase and zymolase in 1.2 M sorbitol, 0.12 M K₂HPO₄, 33 mM citric acid (pH 5.9) for 90 min at 30° C. The membrane is again washed 3 times with sorbital buffer. The cells are then lysed by placing the membrane in methanol for 6 min at −20° C.

After fixation and enzymatic digestion of the cell wall and lysis, the membrane are blocked with 1% BSA in PBS (with several changes). Phosphorylation of the regulatory serines are assessed by using antibodies directed against Phospho IκBα (ser 32) (Cell Signaling 5A5 monoclonal antibody). As a control for expression of the substrate, an antibody against IκBα is also used, and as a control for expression of IKKβ, the blots are also probed for IKKβ. Several different concentrations of primary antibody, and several different detection systems, such as fluorescently labeled 1° or 2° antibodies or alkaline phosphatase linked 1° or 2° antibodies, or horse radish peroxidase 2° antibodies, are tried in order to optimize the system to minimize background while maximizing signal. IκBα should not be phosphorylated in yeast not transformed with IKK, but yeast transformed with IKK should phosphorylate the substrate and, hence, give a positive signal.

Also, as a negative control, yeast containing the IκBα (AA mutant) should not be stained with the Phospho IκBα (ser 32). Another parameter that can be optimized in order to provide a high signal:background ratio, is the time of induction of IKK protein expression. These different strains will be used to optimize the assay conditions.

Finally, as a positive control to test whether the IKK is negatively regulated in the yeast cell, PP2A, all three subunits under antibiotic selection markers, are transformed into the yeast with a galactose promoter, allowing the phosphatase to be induced before the kinase. Induction of PP2A before IKK generates a yeast cell in which the IKK is present but not active. The resulting yeast have much less phosphorylation of IκBα. For these assays, antibodies against PP2A are used in order to assess the expression of PP2A.

### Example IV

The present invention can also be used in an assay to screen a cDNA library from non-stimulated HeLa cells for negative regulators of IKK. A non-stimulated HeLa cell cDNA library is created in a yeast expression vector (Ebrahim Zandi unpublished data). This cDNA library is transformed into yeast cells that were previously transformed with IKK and with wild type IκBα. As in EXAMPLE II described above, the transformants are replica plated, and the yeast is grown on membranes on selective non-inducing medium. After the yeast cells are induced, the transformants are fixed and probed for the presence of IKKβ, IκBα, and Phospho-IκBα (ser 32). The clones that express IKKβ, and IκBα, but are negative for Phospho-IκBα (ser 32) are possible positive clones.

For the positive clones, the plasmid for DNA sequencing is isolated for re-introduction into yeast for protein expression. Identity or homology to known sequences in the data base may suggest the function of the protein which is tested. Whether this protein can maintain IKK, which was previously inactivated by PP2A, in an inactive state can also be tested.

If most of the clones isolated are non-specific phosphatases, an alternate biochemical approach can be used to isolate the negative regulator: two different assays during each purification step. The first assay assesses the ability of fractions to weaken the affinity of IKKγ for the C-terminus of IKKβ. This assay is analyzed by coimmunoprecipitation. The second assay assesses the ability of the fractions to phosphorylate S740 or S750 of IKKβ. To isolate the negative regulator, standard biochemical purification techniques are used including ion exchange chromatography, gel filtration, and ammonium sulfate precipitation to isolate the negative regulator. In addition, a C-terminal peptide of IKKβ to agarose beads to create a matrix for affinity chromatography is attached. Once the negative regulator is purified to near homogeneity, a preparative SDS PAGE gel is run, the bands are cut out, and the proteins are microsequenced.

The putative gene(s) are amplified by PCR using the cDNA library from HeLa cells with primers based on the determined sequence. The gene is cloned into yeast and mammalian cells expression vectors. The protein is expressed in yeast that express IKKα:β:γ to determine if the protein prevents self-activation of IKK. The isolated protein will also be transfected into mammalian cells to see if it inhibits either basal or cytokine-induced IKK activity.

### Example V

The invented composition can also be used to screen for pharmacological therapies to block its activity. This can be accomplished in two ways: first, libraries of small molecule compounds can be tested in in vitro kinase assays to inhibit or further activate the IKK complex made in yeast. For example, partially purified IKK complex from yeast can be incubated with a small molecule prior to testing its activity by in vitro kinase assay. Because this is a simple assay, a large number of compounds can be tested to determine if they inhibit or activate IKK. Second, the yeast system described above in EXAMPLE III, can be used to screen for small molecules that would inhibit or further activate the activity of IKK in situ (yeast).

### Example VI

In another embodiment, the present invention can be used as a source of material for crystallization and X-ray diffraction analysis. The present invention can be used to produce and purify highly homogenous IKK complexes of various compositions in large quantities. Yeast expressing IKK of different compositions can be grown in large quantities (e.g. 10 to 20 liters) and lysed. IKK complexes can be purified by affinity chromatography. This material can be used for producing crystals for X-ray differentiation and structural analysis. Purified IKK can also be used for cryo-EM single particle reconstitution analysis of IKK complex structure at lower resolutions. Because this method requires small amounts of purified proteins, the yeast system disclosed in the present invention can be used to produce various mutant forms of IKK subunits for structure determination. Comparison of wild type and mutant structures of IKK provides information for rational and targeted design of inhibitor and activators for the complex.

Although the foregoing examples have described illustrative embodiments, further uses of the present invention are anticipated. For example, the present invention can also be used to screen for unknown substrates. Different compositions of IKK complexes (α:α:γ, α:β:γ, β:β:γ) may have different substrates. Peptide libraries and/or cDNA expression libraries can be screened to identify specific substrates for a specific IKK composition.

TABLE 1
plasmids for expressing IKK in yeast

| | | | Selection | Subunit | | |
|---|---|---|---|---|---|---|
| Plasmid name | Tag(s) | promoter | marker | of IKK | Mutation | Date |
| pESC leu IKKα | Myc | gal | leucine | IKKα | None | July 1999 |
| pESC his IKKβ | HA | gal | histidine | IKKβ | None | July 1999 |
| pESC trp IKKγ | FLAG | gal | tryptophan | IKKγ | None | July 1999 |
| | 6his | | | | | |
| pES86(+)IKKγ | FLAG | ADH | uracil | IKKγ | None | 15 Nov. 1999 |
| | 6his | | | | | |
| leu(met) IKKγ FL | HA | met | leucine | IKKγ | None | 21 Apr. 2000 |
| leu(met) IKKγΔC300 | HA | met | leucine | IKKγ | C terminus | 21 Apr. 2000 |
| | | | | | Deletion | |
| pESC his IKKβ_{KA} | HA | gal | histidine | IKKβ | Kinase | 16 Jun. 2000 |
| | | | | | defective | |
| PESC his IKKβ_{EEM10} | HA | gal | histidine | IKKβ | Constitutively | 16 Jun. 2000 |
| | | | | | active | |
| pESC ura (met) IKKα | HA | met | uracil | IKKα | None | 30 May 2000 |
| pESC trp (met) IKKβ | HA | met | tryptophan | IKKβ | None | 10 Jul. 2000 |
| pESC trp (met) IKKβ_{KA} | HA | met | tryptophan | IKKβ | Kinase | 10 Jul. 2000 |
| | | | | | defective | |
| pESC trp (met) IKKβ_{EEM10} | HA | met | tryptophan | IKKβ | Constitutively | 10 Jul. 2000 |
| | | | | | active | |
| pESC ura (met) IKKα_{del NBD} | HA | met | uracil | IKKα | Remove NBD | 6 Nov. 2000 |
| pESC trp (met) IKKβ_{del NBD} | HA | met | tryptophan | IKKβ | Remove NBD | 6 Nov. 2000 |
| leu (met) IKKγdel | HA | met | leucine | IKKγ | Remove γ | 3 Jan. 2001 |
| | | | | | interaction | |
| | | | | | domain | |

TABLE 2
Yeast strains

| Name of strain | IKKα plasmid | IKKβ plasmid | IKKγ plasmid(s) | date |
|---|---|---|---|---|
| α1 | pESC leu IKKα | | | 12 Aug. 1999 |
| β1 | | pESC his IKKβ | | 13 Aug. 1999 |
| αγ2 | pESC leu IKKα | | pESC trp IKKγ | 13 Aug. 1999 |
| γ2 | | | pESC trp IKKγ | 13 Aug. 1999 |
| αβγ2 | pESC leu IKKα | pESC his IKKβ | pESC trp IKKγ | 13 Aug. 1999 |
| αβ3 | pESC leu IKKα | pESC his IKKβ | | 13 Aug. 1999 |
| β1 + IKKγ | | pESC his IKKβ | leu(met) IKKγ FL | 6 May 2000 |
| β1 + C300 | | pESC his IKKβ | leu(met) | 6 May 2000 |
| | | | IKKγΔC300 | |
| αβγ9 | pESC ura (met) IKKα | pESC his IKKβ | leu(met) IKKγ FL | 28 Jun. 2000 |
| αβ_{KA}γ | pESC ura (met) IKKα | pESC his IKKβ_{KA} | leu(met) IKKγ FL | 28 Jun. 2000 |
| αβ_{EEM10}γ | pESC ura (met) IKKα | pESC his IKKβ_{EEM10} | leu(met) IKKγ FL | 28 Jun. 2000 |
| α60 | pESC ura (met) IKKα | | | 28 Jul. 2000 |
| β37 | | pESC trp (met) IKKβ | | 20 Jul. 2000 |
| αγ52 | pESC ura (met) IKKα | | leu(met) IKKγ FL | 23 Jul. 2000 |
| βγ65 | | pESC trp (met) IKKβ | leu(met) IKKγ FL | 28 Jul. 2000 |
| αβγ46 | pESC ura (met) IKKα | pESC trp (met) IKKβ | leu(met) IKKγ FL | 23 Jul. 2000 |
| β_{EEM10}γ 74, 75 | | pESC his IKKβ_{EEM10} | | 4 Aug. 2000 |
| αβ_{KA}γ94 | pESC ura (met) IKKα | pESC trp (met) IKKβ_{KA} | leu(met) IKKγ FL | 16 Aug. 2000 |
| αβ100 | pESC ura (met) IKKα | pESC trp (met) IKKβ | | 12 Sep. 2000 |
| γ111 | | | leu(met) IKKγ FL | 12 Sep. 2000 |
| αβ_{EEM10}γ121 | pESC ura (met) IKKα | pESC his IKKβ_{EEM10} | leu(met) IKKγ FL | 12 Sep. 2000 |
| β_{KA}γ135 | | pESC trp (met) IKKβ_{KA} | leu(met) IKKγ FL | 12 Sep. 2000 |
| α_{DEL}β_{WT}γ210 | pESC ura (met) IKKα_{del NBD} | pESC trp (met) IKKβ | leu(met) IKKγ FL | 30 Nov. 2000 |
| and 212 | | | | |
| α_{WT}β_{DEL}γ222 | pESC ura (met) IKKα | pESC trp (met) IKKβ_{del NBD} | leu(met) IKKγ FL | 30 Nov. 2000 |
| α_{DEL}β_{DEL}γ209 | pESC ura (met) IKKα_{del NBD} | pESC trp (met) IKKβ_{del NBD} | leu(met) IKKγ FL | 30 Nov. 2000 |
| α_{DEL}254 | pESC ura (met) IKKα_{del NBD} | | | |
| β_{DEL}233 | | pESC trp (met) IKKβ_{del NBD} | | 30 Nov. 2000 |
| α_{DEL}γ255 | pESC ura (met) IKKα_{del NBD} | | leu(met) IKKγ FL | 30 Nov. 2000 |
| β_{DEL}γ258 | | pESC trp (met) IKKβ_{del NBD} | leu(met) IKKγ FL | 30 Nov. 2000 |
| β_{DEL}γ 297, 298, | | pESC trp (met) IKKβ_{del NBD} | leu(met) IKKγ FL | 11 Jan. 2001 |
| and 299 | | | | |
| αβγ_{DEL}301 | pESC ura (met) IKKα | pESC trp (met) IKKβ | leu (met) IKKγdel | 11 Jan. 2001 |
| βγ_{DEL}303 | | pESC trp (met) IKKβ | leu (met) IKKγdel | 11 Jan. 2001 |
| αγ_{DEL}309 | pESC ura (met) IKKα | | leu (met) IKKγdel | 11 Jan. 2001 |
| γ_{DEL}313 | | | leu (met) IKKγdel | 11 Jan. 2001 |

### REFERENCES

1. Auphan, N., J. A. DiDonato, C. Rosette, A. Helmberg, and M. Karin 1995. Immunosuppression by glucocorticoids: inhibition of NF-kappa B activity through induction of I kappa B synthesis. Science. 270:286-290.
2. Baeuerle, P. A., and D. Baltimore 1988. I kappa B: a specific inhibitor of the NF-kappa B transcription factor. Science. 242:540-546.
3. Baud, V., Z.-G. Liu, B. Bennett, N. Suzuki, Y. Xia, and M. Karin 1999. Signaling by proinflammatory cytokines: oligomerization of TRAF2 and TRAF6 is sufficient for JNK and IKK activation and target gene induction via an amino-terminal effector domain. Genes & Develop. 13:1297-1308.
4. Beg, A. A., and D. Baltimore 1996. An essential role for NF-kappa-B in preventing TNF-alpha-induced cell death. Science. 274:782-784.
5. Beg, A. A., W. C. Sha, R. T. Bronson, S. Ghosh, and D. Baltimore 1995. Embryonic lethality and liver degeneration in mice lacking the RelA component of NF-κB. Nature. 376:167-169.
6. Brach, M. A., R. Hass, M. L. Sherman, H. Gunji, and R. Weichselbaum 1991. Ionizing radiation induces expression and binding activity of the nuclear factor kappa B. J. Clin. Invest. 88:691-695.
7. Christianson, T. W., R. S. Sikorski, M. Dante, J. H. Shero, and P. Hieter 1992. Multifunctional yeast high-copy-number shuttle vectors. Gene. 110:119-122.
8. DiDonato, J. A., M. Hayakawa, D. M. Rothwarf, E. Zandi, and M. Karin 1997. A cytokine-responsive IkappaB kinase that activates the transcription factor NF-kappaB. Nature. 388:548-554.
9. Gill, J. S., and A. J. Windebank 2000. Ceramide initiates NFkappaB-mediated caspase activation in neuronal apoptosis. Neurobiol. Dis. 7:448-461.
10. Gilmore, T. D., M. Koedood, K. A. Piffat, and D. W. White 1996. Rel/NF-kappa-B proteins and cancer. Oncogene. 13:1367-1378.
11. Guttridge, D. C., M. W. Mayo, L. V. Madrid, C. Y. Wang, and A. S. Baldwin, Jr. 2000. NF-kappaB-induced loss of MyoD messenger RNA: possible role in muscle decay and cachexia. Science. 289:2363-2366.
12. Huynh, Q. K., H. Boddupalli, S. A. Rouw, C. M. Koboldt, T. Hall, C. Sommers; S. D. Hauser, J. L. Pierce, R. G. Combs, B. A. Reitz, J. A. Diaz-Collier, R. A. Weinberg, B. L. Hood, B. F. Kilpatrick, and C. T. Tripp 2000. Characterization of the recombinant IKK1/IKK2 heterodimer. J. Biol. Chem. 275:25883-25891.
13. Lee, H. H., H. Dadgostar, Q. Cheng, J. Shu, and G. Cheng 1999. NF-kB-mediated up-regulation of Bcl-x and Bfl-1/A1 is required for CD40 survival signaling in B lymphocytes. Proc. Natl. Acad. Sci. U.S.A. 96:9136-9141.
14. Lee, J. I., and G. J. Burckart 1998. Nuclear factor kappa B: important transcription factor and therapeutic target. J. Clin. Pharm. 38:981-993.
15. Li, N., and M. Karin 1998. Ionizing radiation and short wavelength UV activate NF-kappaB through two distinct mechanisms. Proc. Natl. Acad. Sci. U.S.A. 95:13012-13017.
16. Li, Q., D. VanAntwerp, D. Mercurio, K. F. Lee, and I. M. Verma 1999. Severe liver degeneration in mice lacking the IkB kinase 2 gene. Science. 284:321-325.
17. Liu, Z. G., H. L. Hsu, D. V. Goeddel, and M. Karin 1996. Dissection of TNF receptor 1 effector functions—JNK activation is not linked to apoptosis while NF-kappa-B activation prevents cell death. Cell. 87:565-576.
18. Makris, C., V. L. Godfrey, G. Krahn-Senftleben, T. Takahashi, J. L. Roberts, T. Schwarz, L. Feng, R. S. Johnson, and M. Karin 2000. Female mice heterozygous for IKK gamma/NEMO deficiencies develop a dermatopathy similar to the human X-linked disorder incontinentia pigmenti. Mol. Cell. 5:969-979.
19. May, M. J., F. D'Acquisto, L. A. Madge, J. Glockner, J. S. Pober, and S. Ghosh 2000. Selective inhibition of NF-kappaB activation by a peptide that blocks the interaction of NEMO with the IkappaB kinase complex. Science. 289:1550-1554.
20. Mercurio, F., H. Zhu, B. W. Murray, A. Shevchenko, B. L. Bennett, J. Li, D. B. Young, M. Barbosa, M. Mann, A. Manning, and A. Rao 1997. IKK-1 and IKK-2: cytokine-activated IkappaB kinases essential for NF-kappaB activation. Science. 278:860-866.
21. Mosialos, G. 1997. The role of Rel/NF-kappa B proteins in viral oncogenesis and the regulation of viral transcription. Sem. Cancer Biol. 8:121-129.
22. Mumberg, D., R. Muller, and M. Funk 1994. Regulatable promoters of Saccharomyces cerevisiae: comparison of transcriptional activity and their use for heterologous expression. Nuc. Acids Res. 22:5767-5768.
23. Osborn, L., S. Kunkel, and G. J. Nabel 1989. Tumor necrosis factor alpha and interleukin 1 stimulate the human immunodeficiency virus enhancer by activation of the nuclear factor kappa B. Proc. Natl. Acad. Sci. U.S.A. 86:2336-2340.
24. Pahl, H. L. 1999. Activators and target genes of R^{e1}/NF-kB transcription factors. Oncogene. 18:6853-6866.
25. Ray, A., and K. E. Prefontaine 1994. Physical association and functional antagonism between the p65 subunit of transcription factor NF-kappa B and the glucocorticoid receptor. Proc. Natl. Acad. Sci. U.S.A. 91:752-756.
26. Roshak, A. K., J. R. Jackson, K. McGough, M. Chabot-Fletcher, E. Mochan, and L. A. Marshall 1996. Manipulation of distinct NF-kB proteins alters interleukin-1-induced human rheumatoid synovial fibroblast prostaglandin E2 formation. J. Biol. Chem. 271:31496-31501.
27. Rothwarf, D. M., E. Zandi, G. Natoli, and M. Karin 1998. MK-gamma is an essential regulatory subunit of the 1 kB kinase complex. Nature. 395:297-300.
28. Rudolph, D., W.-C. Yeh, A. Wakeham, B. Rudolph, D. Nallainathan, J. Potter, A. J. Elia, and T. W. Mak 2000. Severe liver degeneration and lack of NF-κB activation in NEMO/IKKγ-deficient mice. Genes & Develop. 14:854-862.
29. Sakurada, S., T. Kato, and T. Okamoto 1996. Induction of cytokines and ICAM-1 by proinflammatory cytokines in primary rheumatoid synovial fibroblasts and inhibition by N-acetyl-L-cysteine and aspirin. Int. Immunol. 8:1483-1493.
30. Schreck, R., K. Albermarm, and P. A. Baeuerle 1992. Nuclear factor kappa B: an oxidative stress-responsive transcription factor of eukaryotic cells (a review). Free Radic. Res. Commun. 17:221-237.
31. Sen, R., and D. Baltimore 1986. Inducibility of kappa immunoglobulin enhancer-binding protein NF-kappa B by a posttranslational mechanism. Cell. 47:921-928.
32. Siebenlist, U., G. Franzoso, and K. Brown 1994. Structure, regulation and function of NF-kappa B. Annu. Rev. Cell Biol. 10:405-455.
33. Stehlik, C., R. de Martin, I. Kumabashiri, J. A. Schmid, B. R. Binder, and J. Lipp 1998. Nuclear factor (NF)-kB-regulated X-chromosome-linked iap gene expression protects endothelial cells from tumor necrosis factor-induced apoptosis. J. Exp. Med. 188:211-216.
34. VanAntwerp, D. J., S. J. Martin, T. Kafri, D. R. Green, and I. M. Verma 1996. Suppression of TNF-alpha-induced apoptosis by NF-kappa-B. Science. 274:787-789.
35. VanAntwerp, D. J., S. J. Martin, I. M. Verna, and D. R. Green 1998. Inhibition of TNF-induced apoptosis by NF-kappa B. Trends Cell Biol. 8:107-111.
36. Wang, C. Y., M. W. Mayo, and A. S. Baldwin 1996. TNF- and cancer therapy-induced apoptosis—potentiation by NF-kappa B. Science. 274:784-787.
37. Wang, C. Y., M. W. Mayo, R. G. Komeluk, D. V. Goeddel, and A. S. Baldwin, Jr. 1998. NF-kappa B antiapoptosis: induction of TRAF1 and TRAF2 and c-IAP1 and c-IAP2 to suppress caspase-8 activation. Science. 281:1680-1683.
38. Weber, C. K., S. Liptay, T. Wirth, G. Adler, and R. M. Schmid 2000. Suppression of NF-kappaB activity by sulfasalazine is mediated by direct inhibition of IkappaB kinases alpha and beta. Gastroenterology. 119:1209-1218.
39. Yin, M.-J., Y. Yamamoto, and R. B. Gaynor 1998. The anti-inflammatory agents aspirin and salicylate inhibit the activity of I(kappa)B kinase-beta. Nature. 396:77-80.
40. You, M., P.-T. Ku, R. Hrdlickova, and H. R. Bose, Jr. 1997. ch-IAP1, a member of the inhibitor-of-apoptosis protein family, is a mediator of the antiapoptotic activity of the v-Rel oncoprotein. Mol. Cell. Biol. 17:7328-7341.
41. Yujiri, T., M. Ware, C. Widmann, R. Oyer, D. Russell, E. Chan, Y. Zaitsu, P. Clarke, K. Tyler, Y. Oka, G. R. Fanger, P. Henson, and G. L. Johnson 2000. MEK kinase 1 gene disruption alters cell migration and c-Jun NH₂-terminal kinase regulation but does not cause a measurable defect in NF-κB activation. Proc. Natl. Acad. Sci. U.S.A. 97:7272-7277.
42. Zandi, E., Y. Chen, and M. Karin 1998. Direct phosphorylation of IkappaB by IKKalpha and IKKbeta: discrimination between free and NF-kappaB-bound substrate. Science. 281:1360-1363.
43. Zandi, E., and M. Karin 1999. Bridging the gap: composition, regulation, and physiological function of the IkB kinase complex. Mol. Cell. Biol. 19:4547-4551.
44. Zandi, E., D. M. Rothwarf, M. Delhase, M. Hayakawa, and M. Karin 1997. The IkappaB kinase complex (IKK) contains two kinase subunits, IKKalpha and IKKbeta, necessary for IkappaB phosphorylation and NF-kappaB activation. Cell. 91:243-252.
45. Zhang, S. Q., A. Kovalenko, G. Cantarella, and D. Wallach 2000. Recruitment of the IKK signalosome to the p55 TNF receptor: RIP and A20 bind to NEMO (IKKγ) upon receptor stimulation. Irrununity. 12:301-311.
46. Watson, J. D., Hopkins, N. H., Roberts, L. W., Steitz, L. A., and Weiner, A. M. 1987. In Molecular Biology of the Gene, Vola 1, pp. 550-594. Benjamin/Cummings, Menlo Park, Calif.

We claim:

1. A method for reconstituting IKK in yeast comprising the steps of:
a. subcloning IKK subunit genes into yeast expression vectors;
b. transforming said yeast expression vectors into yeast;
c. growing said yeast in a selective liquid media; and
d. controllably inducing the expression of said IKK subunits by means of inducible promoters.

2. The method of claim 1, further comprising the steps of:
a. lysing said yeast;
b. extracting said IKK protein; and
c. purifying said IKK protein.

3. The method of claim 1, wherein said yeast expression vectors contain a selection marker.

4. The method of claim 2, wherein said selection marker is leucine, histidine, tryptophan, or uracil.

5. The method of claim 1, wherein said yeast expression vectors contain a tag.

6. The method of claim 1.a, wherein said tag is myc, HA, or FLAG 6his.

7. The method of claim 1, wherein said yeast expression vectors contain an inducible promoter or a constitutive promoter.

8. The method of claim 1.a, wherein said inducible promoter is methionine or galactose.

9. The method of claim 1.a, wherein said constitutive promoter is alcohol dehydrogenase.

10. The method of claim 1, wherein said IKK subunit is IKKα.

11. The method of claim 1, wherein said IKK subunit is IKKβ.

12. The method of claim 1, wherein said IKK subunit is IKKγ.

13. The method of claim 1, wherein said IKK subunits are a combination of IKKα, IKKβ, and IKKγ.

14. The method of claim 1.a, 1.a or 1.a wherein said IKKα and IKKβ subunits are subcloned into pESC ura or pESC trp vectors wherein a galactose promoter region is replaced with a met promoter from a leu(met) vector.

15. The method of claim 1.a or 1.a, wherein said IKKγ subunit is subcloned into said leu(met) vector.

16. The method of claim 1.a or 1.a, wherein said IKKγ subunit is subcloned into the pES 86(+) expression vector wherein constitutive expression is induced under the alcohol dehydrogenase promoter.

17. The method of claim 1, wherein said yeast is Saccharomyces cerevisiae.

18. The method of claim 1, wherein said IKK is mammalian IKK.

19. The method of claim 1.a, wherein said mammalian IKK is human IKK.

20. The method of claim 1, wherein said vectors are plasmids, small yeast chromosomes or cosmids.

21. The method of claim 1, wherein said selective liquid media is an non-inducing drop-out media.

22. The method of claim 1, wherein said purified IKK protein is substantially homologous to IKK isolated from wild-type cells.

23. The method of claim 1, wherein said purified IKK protein is mutated.

24. A heterologously expressed IKK complex, wherein said IKK is expressed by yeast.

25. The composition of claim 24, wherein said IKK complex is comprised of IKKα, IKKβ, and IKKγ subunits.

26. The composition of claim 24, wherein said IKK complex is produced by the method of claim 1.

27. A heterologously expressed IKK complex, wherein said IKKγ protein subunit regulates phosphorylation of serine residues in the activation of T loop kinase domain of IKK catalytic subunits.

28. The method of claim 27, wherein said IKK complex is activated by the dephosphorylation of γBD serines.

29. A yeast cell containing an expressible copy of a gene encoding a subunit of IKK.

30. The yeast cell of claim 1.a which is transformed with a yeast expression vector which contains the expressible copy of the gene encoding IKKα, IKKβ, or IKKγ.

31. The yeast cell of claim 1.a which is transformed by the method of claim 1.

32. A method for identifying upstream regulators of IKK complex, comprising the steps of:
a. mutating the genes of one or more said IKK subunits;
b. subcloning genes for IKK subunits into yeast expression vectors;
c. transforming said yeast expression vectors into yeast;
d. growing said yeast in a selective liquid media;
e. controllably inducing the expression of said IKK subunits by means of inducible promoters;
f. lysing said yeast;
g. extracting said IKK protein;
h. purifying said IKK protein; and
i. comparing kinase activity of said IKK protein with wild type IKK.

33. The method of claim 32, wherein said mutation is on a binding domain.

34. The method of claim 1.a, wherein said mutation mimics the biochemical characteristics of said binding site when bound.

35. The method of claim 1.a, wherein said mutation prevents binding at said domain site.

36. The method of claim 32, wherein said mutation changes serines to alanines.

37. The method of claim 32, wherein said mutation changes serines to glutamic acid.

38. A method for assaying IKK activity in situ in yeast comprising the steps of:
a. subcloning genes for IKK subunits into first yeast expression vectors;
b. transforming said first yeast expression vectors into yeast;
c. subcloning HeLa cell cDNA into second yeast expression vectors;
d. transforming said second yeast expression vectors into said yeast;
e. replica plating said yeast;
f. growing said yeast on membranes on selective non-inducing medium
g. inducing said yeast to produce IKK protein;
h. fixing said IKK protein;
i. probing said IKK protein with IKKβ, IκBα, and Phospho-IκBα (ser 32); and
j. isolate on said membranes clones positive for IKKβ and IκBα and negative for Phospho-IκBα (ser 32).

39. The method of claim 1.a, further comprising the step of sequencing said positive clones.

40. The method of claim 1.a, further comprising the steps of:
a. transforming said positive clone into yeast;
b. growing said yeast in a selective liquid media;
c. controllably inducing the expression of said clones by means of inducible promoters.
